(19) United States
(12) Patent Application Publication
(10) Pub. No.: US 2026/0137936 A1
(43) Pub. Date: May 21, 2026

(54) SYSTEMS AND METHODS FOR NEUROMODULATION USING DELAYED DISCHARGE PULSE WAVEFORMS

(71) Applicant: Advanced Neuromodulation Systems, Inc., Plano, TX (US)

(72) Inventor: Hyun-Joo PARK, Frisco, TX (US)

(73) Assignee: Advanced Neuromodulation Systems, Inc., Plano, TX (US)

(21) Appl. No.: 19/344,256

(22) Filed: Sep. 29, 2025

Related U.S. Application Data
(60) Provisional application No. 63/704,543, filed on Oct. 7, 2024.

(51) Int. Cl. A61N 1/36; A61N 1/372

(52) U.S. Cl. CPC A61N 1/36062; A61N 1/36071; A61N 1/36164; A61N 1/36175; A61N 1/36178; A61N 1/37247; A61N 1/37264

(57) ABSTRACT

In some embodiments, a method of providing a neurostimulation therapy to a patient comprises: generating, by an implantable pulse generator (IPG), electrical pulses comprising a plurality of pulses having a uniform pulse width over time and a pulse amplitude that changes over time, and applying the generated electrical pulses to neural tissue of the patient to inhibit neural activity of the patient. The pulse amplitude may change sinusoidally, linearly, or exponentially over time.

### CROSS-REFERENCE TO RELATED APPLICATIONS

The present application claims the benefit of priority from U.S. Provisional Application No. 63/704,543, filed Oct. 7, 2024 and entitled “SYSTEM AND METHODS FOR NEUROMODULATION USING DELAYED DISCHARGE PULSE WAVEFORMS,” the contents of which are incorporated herein by reference in its entirety. In addition, the present application is related to U.S. patent application Ser. No. 17/980,325, filed Nov. 3, 2022 and entitled “SYSTEM AND METHODS TO DELIVER HYPERPOLARIZING WAVEFORM,” which claims the benefit of priority from U.S. Provisional Application No. 63/276,020, filed Nov. 5, 2021 and entitled “SYSTEM AND METHODS TO DELIVER HYPERPOLARIZING WAVEFORM,” the contents of which are incorporated herein by reference in their entirety.

### BACKGROUND

Implantable medical devices have improved medical care for patients with certain types of chronic illnesses and disorders. For example, implantable cardiac devices improve cardiac function in patients with heart disease thereby raising quality of life and reducing morality rates. Implantable neurostimulators can provide pain reduction for chronic pain patients and reduce motor difficulties in patients with Parkinson's disease and other movement disorders. A variety of other medical devices are proposed and are in development to treat other disorders in a wide range of patients.

Neural activity can be influenced by electrical energy that is supplied from a stimulation system pulse generator or other waveform generator. Various patient perceptions and/or neural functions can be promoted, disrupted, or otherwise modified by applying electrical pulses to target sites (the spinal cord, dorsal root ganglia, peripheral nerves, cortical locations, and deep brain locations as examples). For example, spinal cord stimulation has been known to reduce pain levels for chronic pain patients for many years. Also, medical researchers and clinicians have attempted to treat various neurological conditions using electrical stimulation to control or affect brain functions. For example, Deep Brain Stimulation (DBS) may reduce some of the symptoms associated with Parkinson's disease.

A stimulation system pulse generator may be provided in various configurations, such as an implanted pulse generator (IPG). A typical IPG configuration comprises a surgically implanted, internally-powered pulse generator and multi-electrode lead. The implanted pulse generator may commonly be encased in a hermetically sealed housing and surgically implanted in a subclavicular location. An electrode assembly may be implanted to deliver stimulation signals to a stimulation site. The electrode assembly is coupled to the pulse generator via biocompatibly sealed lead wires. A power source, such as a battery, is contained within the housing of the pulse generator.

### SUMMARY

In some embodiments, a method of providing a neurostimulation therapy to a patient comprises: generating electrical pulses, by an IPG, comprising respective bursts of a plurality of anodic pulses with each anodic pulse being separated by a time gap, wherein (1) the plurality of anodic pulses comprise successively increasing charges; (2) the plurality of anodic pulses are charge limited to be sub-threshold; (3) each burst of anodic pulses is followed by a discharge phase of intermittent time periods to discharge charge build up from the anodic pulses; and (4) each successive intermittent time period increases in time through the discharge phase to avoid action potential (AP) generation; and applying the generated electrical pulses to neural tissue of the patient to inhibit neural activity of the patient.

In some embodiments, a method of providing a neurostimulation therapy to a patient comprises generating, by an IPG, electrical pulses comprising a plurality of pulses having a uniform pulse width over time and a pulse amplitude that changes over time. In some embodiments, the plurality of pulses comprise a plurality of anodic pulses, a plurality of cathodic pulses, or a combination thereof. In some embodiments, the pulse amplitude may change sinusoidally, linearly, or exponentially over time. The method also comprises applying the generated electrical pulses to neural tissue of the patient to inhibit neural activity of the patient. The electical pulses may be configured to treat a medical condition of the patient. As a non-lmiting example, the medical condition may include pain of the patient, such as pain that may be treated with spinal cord stimulation (SCS). In some embodiments, the generated electrical pulses are applied to dorsal fibers of the spinal cord. For example, the electrical pulses may be configured to suppress nociceptive projection neurons in a superficial lamina of a dorsal horn of a spine of the patient.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

FIG. 1 depicts a neurostimulation system according to some embodiments.

FIG. 2 depicts a computer system according to some embodiments.

FIG. 3 depicts a system for monitoring and/or managing neurostimulation therapies for patients according to some embodiments.

FIG. 4 depicts an inhibiting waveform for neurostimulation according to some embodiments.

FIG. 5 depicts defining a neurostimulation therapy for a patient according to some embodiments.

FIG. 6 is a diagram illustrating an example waveform exhibiting a gradual increase of pulse amplitude in accordance with some embodiments.

FIG. 7 is a diagram illustrating an example waveform exhibiting a gradual decrease of pulse amplitude in accordance with some embodiments.

FIG. 8 is a diagram illustrating an example waveform exhibiting a gradual increase of pulse amplitude followed by gradual decrease of the pulse amplitude in accordance with some embodiments.

FIG. 9 is a diagram illustrating an example waveform having an anodic pulse train with gradual increase of the pulse amplitude followed by a cathodic pulse train with gradual increase of the pulse amplitude in accordance with some embodiments.

FIG. 10 is a diagram illustrating an example waveform having an anodic pulse train with gradual increase of the pulse amplitude followed by a cathodic pulse train with gradual decrease of the pulse amplitude in accordance with some embodiments.

FIG. 11 is a diagram illustrating an example waveform having an anodic pulse train with gradual increase of the pulse amplitude followed by a cathodic pulse train with a gradual decrease of the pulse amplitude followed by a gradual increase in pulse amplitude in accordance with some embodiments.

FIG. 12 is a block diagram illustrating an example user interface (UI) for generating stimulation waveforms in accordance with some embodiments.

FIG. 13 is a flow diagram of an example method for providing a neurostimulation therapy to a patient in accordance with some embodiments.

### DETAILED DESCRIPTION

Neurostimulation systems are devices that generate electrical pulses and deliver the pulses to neural tissue of a patient to treat a variety of disorders. One category of neurostimulation systems is deep brain stimulation (DBS). In DBS, pulses of electrical current are delivered to target regions of a subject's brain, for example, for the treatment of movement and effective disorders such as Parkinson's disease and essential tremor. Another category of neurostimulation systems is spinal cord stimulation (SCS) which is often used to treat chronic pain such as Failed Back Surgery Syndrome (FBSS) and Complex Regional Pain Syndrome (CRPS). Dorsal root ganglion (DRG) stimulation is another example of a neurostimulation therapy in which electrical stimulation is provided to the dorsal root ganglion structure that is just outside of the epidural space. DRG stimulation is generally used to treat chronic pain.

Neurostimulation systems generally include a pulse generator and one or more leads. A stimulation lead includes a lead body of insulative material that encloses wire conductors. The distal end of the stimulation lead includes multiple electrodes or contacts for application of electrical pulses to patient tissue. The electrodes or contacts are electrically coupled to the wire conductors of a respective stimulation lead. The proximal end of the lead body includes multiple terminals (also electrically coupled to the wire conductors) that are adapted to receive electrical pulses. In DBS systems, the distal end of the stimulation lead is implanted within the brain tissue to deliver the electrical pulses. The stimulation leads are then tunneled to another location within the patient's body to be electrically connected with a pulse generator or, alternatively, to an “extension.” The pulse generator is typically implanted in the patient within a subcutaneous pocket created during the implantation procedure.

The pulse generator is typically implemented using a metallic housing (or can) that encloses circuitry for generating the electrical stimulation pulses, control circuitry, communication circuitry, a rechargeable or primary cell battery, etc. The pulse generating circuitry is coupled to one or more stimulation leads through electrical connections provided in a “header” of the pulse generator. Specifically, feedthrough wires typically exit the metallic housing and enter into a header structure of a moldable material. Within the header structure, the feedthrough wires are electrically coupled to annular electrical connectors. The header structure holds the annular connectors in a fixed arrangement that corresponds to the arrangement of terminals on the proximal end of a stimulation lead.

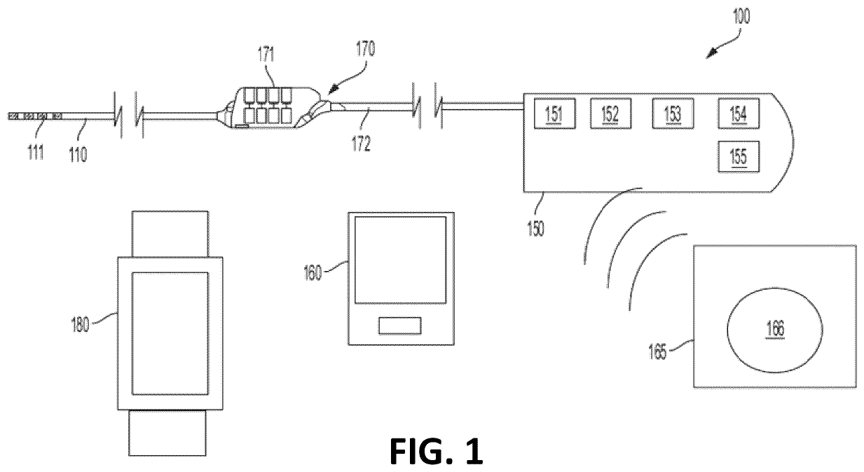

Stimulation system 100 is shown in FIG. 1 according to some embodiments. Stimulation system 100 generates electrical pulses for application to tissue of a patient to treat one or more disorders of the patient. System 100 includes an implantable pulse generator (IPG) 150 that is adapted to generate electrical pulses for application to tissue of a patient. Examples of commercially available implantable pulse generators include the PROCLAIM XR™ and INFINITY™ implantable pulse generators (available from Abbott Laboratories, Plano Texas). Commercially available IPGs may be adapted (using suitable software instructions, programmable parameters, logic circuits, other circuits, and/or the like) according to the disclosures in this application. Alternatively, system 100 may include an external pulse generator (EPG) positioned outside the patient's body. IPG 150 typically includes a metallic housing (or can) that encloses a controller 151, pulse generating circuitry 152, a battery 153, far-field and/or near field communication circuitry 154 (e.g., BLUETOOTH communication circuitry), sensing circuitry 155, and other appropriate circuitry and components of the device. Controller 151 typically includes a microcontroller or other suitable processor for controlling the various other components of the device. Software code is typically stored in memory of IPG 150 for execution by the microcontroller or processor to control the various components of the device.

IPG 150 may comprise one or more attached extension components 170 or be connected to one or more separate extension components 170. Alternatively, one or more stimulation leads 110 may be connected directly to IPG 150. Within IPG 150, electrical pulses are generated by pulse generating circuitry 152 and are provided to switching circuitry. The switching circuit connects to output wires, metal ribbons, traces, lines, or the like (not shown) from the internal circuity of pulse generator 150 to output connectors (not shown) of pulse generator 150 which are typically contained in the “header” structure of pulse generator 150. Commercially available ring/spring electrical connectors are frequently employed for output connectors of pulse generators (e.g., “Bal-Seal” connectors). The terminals of one or more stimulation leads 110 are inserted within connector portion 171 for electrical connection with respective connectors or directly within the header structure of pulse generator 150. Thereby, the pulses originating from IPG 150 are conducted to electrodes 111 through wires contained within the lead body of lead 110. The electrical pulses are applied to tissue of a patient via electrodes 111.

For implementation of the components within IPG 150, a processor and associated charge control circuitry for an implantable pulse generator is described in U.S. Pat. No. 7,571,007, which is incorporated herein by reference. Circuitry for recharging a rechargeable battery of an implantable pulse generator using inductive coupling and external charging circuits are described in U.S. Pat. No. 7,212,110, which is incorporated herein by reference.

An example and discussion of “constant current” pulse generating circuitry is provided in U.S. Patent Publication No. 2006/0170486 which is incorporated herein by reference. One or multiple sets of such circuitry may be provided within IPG 150. Different pulses on different electrodes may be generated using a single set of pulse generating circuitry using consecutively generated pulses according to a “multi-stimset program” as is known in the art. Alternatively, multiple sets of such circuitry may be employed to provide pulse patterns that include simultaneously generated and delivered stimulation pulses through various electrodes of one or more stimulation leads as is also known in the art. Various sets of parameters may define the pulse characteristics and pulse timing for the pulses applied to various electrodes as is known in the art. Although constant current pulse generating circuitry is contemplated for some embodiments, any other suitable type of pulse generating circuitry may be employed such as constant voltage pulse generating circuitry.

Stimulation lead(s) 110 may include a lead body of insulative material about a plurality of conductors within the material that extend from a proximal end of lead 110 to its distal end. The conductors electrically couple a plurality of electrodes 111 to a plurality of terminals (not shown) of lead 110. The terminals are adapted to receive electrical pulses and the electrodes 111 are adapted to apply stimulation pulses to tissue of the patient. Also, sensing of physiological signals may occur through electrodes 111, the conductors, and the terminals. Additionally or alternatively, various sensors (not shown) may be located near the distal end of stimulation lead 110 and electrically coupled to terminals through conductors within the lead body 172. Stimulation lead 110 may include any suitable number and type of electrodes 111, terminals, and internal conductors.

External controller device 160 is a device that permits the operations of IPG 150 to be controlled by a user after IPG 150 is implanted within a patient. Also, multiple controller devices may be provided for different types of users (e.g., the patient or a clinician). Controller device 160 can be implemented by utilizing a suitable handheld processor-based system that possesses wireless communication capabilities. Software is typically stored in memory of controller device 160 to control the various operations of controller device 160. The interface functionality of controller device 160 is implemented using suitable software code for interacting with the user and using the wireless communication capabilities to conduct communications with IPG 150. One or more user interface screens may be provided in software to allow the patient and/or the patient's clinician to control operations of IPG 150 using controller device 160. In some embodiments, commercially available devices such as Apple IOS devices are adapted for use as controller device 160 by include one or more “apps” that communicate with IPG 150 using, for example, BLUETOOTH communication.

Controller device 160 preferably provides one or more user interfaces to allow the user to operate IPG 150 according to one or more stimulation programs to treat the patient's disorder(s). Each stimulation program may include one or more sets of stimulation parameters including pulse amplitude, pulse width, pulse frequency or inter-pulse period, pulse repetition parameter (e.g., number of times for a given pulse to be repeated for respective stimset during execution of program), etc.

Controller device 160 may permit programming of IPG 150 to provide a number of different stimulation patterns or therapies to the patient as appropriate for a given patient and/or disorder. Examples of different stimulation therapies include conventional tonic stimulation (continuous train of stimulation pulses at a fixed rate), BurstDR stimulation (burst of pulses repeated at a high rate interspersed with quiescent periods with or without duty cycling), “high frequency” stimulation (e.g., a continuous train of stimulation pulses at 10,000 Hz), noise stimulation (series of stimulation pulses with randomized pulse characteristics such as pulse amplitude to achieve a desired frequency domain profile). Any suitable stimulation pattern or combination thereof can be provided by IPG 150 according to some embodiments. Controller device 160 communicates the stimulation parameters and/or a series of pulse characteristics defining the pulse series to be applied to the patient to IPG 150 to generate the desired stimulation therapy.

Examples of suitable therapies include tonic stimulation (in which a fixed frequency pulse train) is generated, burst stimulation (in which bursts of multiple high frequency pulses) are generated which in turn are separated by quiescent periods, “high frequency” stimulation, multi-frequency stimulation, and noise stimulation. Descriptions of respective neurostimulation therapies are provided in the following publications: (1) Schu S., Slotty P. J., Bara G., von Knop M., Edgar D., Vesper J., “A Prospective, Randomised, Double-blind, Placebo-controlled Study to Examine the Effectiveness of Burst Spinal Cord Stimulation Patterns for the Treatment of Failed Back Surgery Syndrome,” Neuromodulation 2014; 17: 443-450; (2) Al-Kaisy Al, Van Buyten J P, Smet I, Palmisani S, Pang D, Smith T., “Sustained effectiveness of 10 kHz high-frequency spinal cord stimulation for patients with chronic, low back pain: 24-month results of a prospective multicenter study,” Pain Med. 2014 March;15(3):347-54; and (3) Sweet, Badjatiya, Tan D., Miller, “Paresthesia-Free High-Density Spinal Cord Stimulation for Postlaminectomy Syndrome in a Prescreened Population: A Prospective Case Series,” Neuromodulation. 2016 April;19(3): 260-7. Noise stimulation is described in U.S. Pat. No. 8,682,441. Burst stimulation is described in U.S. Pat. No. 8,224,453 and U.S. Published Application No. 2006/0095088. A “coordinated reset” pulse pattern is applied to neuronal subpopulation/target sites to desynchronize neural activity in the subpopulations. Coordinated reset stimulation is described, for example, by Peter A. Tass et al in “Coordinated Reset has Sustained After Effects nn Parkinsonian Monkeys,” Annals of Neurology, Vol. 7, Iss. 5, pages 816-820, November 2012, which is incorporated herein by reference. The electrical pulses in a coordinated reset pattern are generated in bursts of pulses with respective bursts being applied to tissue of the patient using different electrodes in a time-offset manner. The time-offset is selected such that the phase of the neural-subpopulations are reset in a substantially equidistant phase-offset manner. By resetting neuronal subpopulations in this manner, the population will transition to a desynchronized state by the interconnectivity between the neurons in the overall neuronal population. All of these references are incorporated herein by reference.

For implementation of the components within IPG 150, a processor and associated charge control circuitry for an implantable pulse generator is described in U.S. Pat. No. 7,571,007, which is incorporated herein by reference. Circuitry for recharging a rechargeable battery of an implantable pulse generator using inductive coupling and external charging circuits are described in U.S. Pat. No. 7,212,110, which is incorporated herein by reference.

IPG 150 modifies its internal parameters in response to the control signals from controller device 160 to vary the stimulation characteristics of stimulation pulses transmitted through stimulation lead 110 to the tissue of the patient. Neurostimulation systems, stimsets, and multi-stimset programs are discussed in PCT Publication No. WO 2001/093953, and U.S. Pat. No. 7,228,179, which are incorporated herein by reference.

External charger device 165 may be provided to recharge battery 153 of IPG 150 according to some embodiments when IPG 150 includes a rechargeable battery. External charger device 165 comprises a power source and electrical circuitry (not shown) to drive current through coil 166. The patient places the primary coil 166 against the patient's body immediately above the secondary coil (not shown), i.e., the coil of the implantable medical device. Preferably, the primary coil 166 and the secondary coil are aligned in a coaxial manner by the patient for efficiency of the coupling between the primary and secondary coils. In operation during a charging session, external charger device 165 generates an AC-signal to drive current through coil 166 at a suitable frequency. Assuming that primary coil 166 and secondary coil are suitably positioned relative to each other, the secondary coil is disposed within the magnetic field generated by the current driven through primary coil 166. Current is then induced by a magnetic field in the secondary coil. The current induced in the coil of the implantable pulse generator is rectified and regulated to recharge the battery of IPG 150. IPG 150 may also communicate status messages to external charging device 165 during charging operations to control charging operations. For example, IPG 150 may communicate the coupling status, charging status, charge completion status, etc.

System 100 may include external wearable device 180 such as a smartwatch or health monitor device. Wearable device 180 may be implemented using commercially available devices such as FITBIT VERSA SMARTWATCH™, SAMSUNG GALAXY SMARTWATCH™, and APPLE WATCH™ devices with one or more apps or appropriate software to interact with IPG 150 and/or controller device 160. In some embodiments, wearable device 180, controller device 160, and IPG 150 conduct communications using BLUETOOTH communications.

Wearable device 180 monitors activities of the patient and/or senses physiological signals. Wearable device 180 may track physical activity and/or patient movement through accelerometers. Wearable device 180 may monitory body temperature, heart rate, electrocardiogram activity, blood oxygen saturation, and/or the like. Wearable device 180 may monitor sleep quality or any other relevant health related activity.

Wearable device 180 may provide one or more user interface screens to permit the patient to control or otherwise interact with IPG 150. For example, the patient may increase or decrease stimulation amplitude, change stimulation programs, turn stimulation on or off, and/or the like using wearable device 180. Also, the patient may check the battery status of other implant status information using wearable device 180.

Wearable device 180 may include one or more interface screens to receive patient input. In some embodiments, wearable device 180 and/or controller device 160 are implemented (individually or in combination) to provide an electronic patient diary function. The patient diary function permits the patient to record on an ongoing basis the health status of the patient and the effectiveness of the therapy for the patient. In some embodiments as discussed herein, wearable device 180 and/or controller device 160 enable the user to indicate the current activity of the patient, the beginning of an activity, the completion of an activity, the ease or quality of patient's experience with a specific activity, the patient's experience of pain, the patient's experience of relief from pain by the stimulation, or any other relevant indication of patient health by the patient.

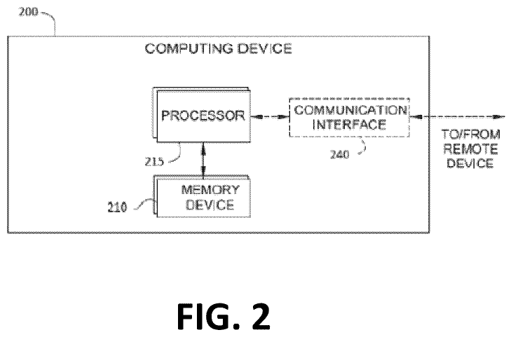

FIG. 2 is a block diagram of one embodiment of a computing device 200 that may be used to according to some embodiments. Computing device 200 may be used to implement external controller device 160, wearable device 180, remote care management servers, or other computing system according to some embodiments.

Computing device 200 includes at least one memory device 210 and a processor 215 that is coupled to memory device 210 for executing instructions. In some embodiments, executable instructions are stored in memory device 210. In some embodiments, computing device 200 performs one or more operations described herein by programming processor 215. For example, processor 215 may be programmed by encoding an operation as one or more executable instructions and by providing the executable instructions in memory device 210.

Processor 215 may include one or more processing units (e.g., in a multi-core configuration). Further, processor 215 may be implemented using one or more heterogeneous processor systems in which a main processor is present with secondary processors on a single chip. In another illustrative example, processor 215 may be a symmetric multi-processor system containing multiple processors of the same type. Further, processor 215 may be implemented using any suitable programmable circuit including one or more systems and microcontrollers, microprocessors, reduced instruction set circuits (RISC), application specific integrated circuits (ASIC), programmable logic circuits, field programmable gate arrays (FPGA), and any other circuit capable of executing the functions described herein.

In the illustrated embodiment, memory device 210 is one or more devices that enable information such as executable instructions and/or other data to be stored and retrieved. Memory device 210 may include one or more computer readable media, such as, without limitation, dynamic random access memory (DRAM), read-only memory (ROM), electrically erasable programmable read-only memory (EEPROM), static random access memory (SRAM), a solid state disk, and/or a hard disk. Memory device 210 may be configured to store, without limitation, application source code, application object code, source code portions of interest, object code portions of interest, configuration data, execution events and/or any other type of data.

Computing device 200, in the illustrated embodiment, includes a communication interface 240 coupled to processor 215. Communication interface 240 communicates with one or more remote devices, such as a clinician or patient programmer. To communicate with remote devices, communication interface 240 may include, for example, a wired network adapter, a wireless network adapter, a radio-frequency (RF) adapter, and/or a mobile telecommunications adapter.

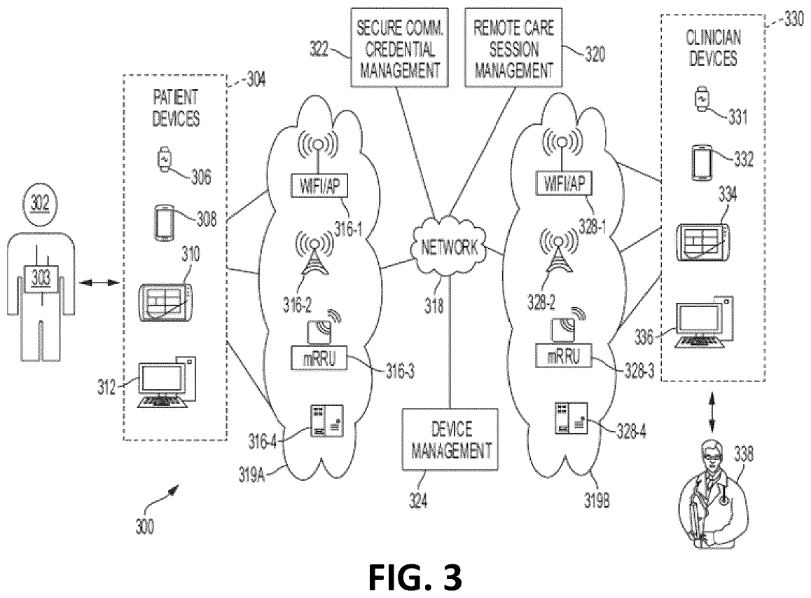

FIG. 3 depicts a network environment 300 for remote management of patient care. One or more embodiments of a remote care therapy application or service may be implemented in network environment 300, as described herein. In general, “remote care therapy” may involve any care, biomedical monitoring, or therapy that may be provided by a clinician, a medical professional or a healthcare provider, and/or their respective authorized agents (including digital/virtual assistants), with respect to a patient over a communications network while the patient and the clinician/provider are not in close proximity to each other (e.g., not engaged in an in-person office visit or consultation). Accordingly, in some embodiments, a remote care therapy application may form a telemedicine or a telehealth application or service that not only allows healthcare professionals to use electronic communications to evaluate, diagnose and treat patients remotely, thereby facilitating efficiency as well as scalability, but also provides patients with relatively quick and convenient access to diversified medical expertise that may be geographically distributed over large areas or regions, via secure communications channels as described herein.

Network environment 300 may include any combination or sub-combination of a public packet-switched network infrastructure (e.g., the Internet or worldwide web, also sometimes referred to as the “cloud”), private packet-switched network infrastructures such as Intranets and enterprise networks, health service provider network infrastructures, and the like, any of which may span or involve a variety of access networks, backhaul and core networks in an end-to-end network architecture arrangement between one or more patients, e.g., patient(s) 302, and one or more authorized clinicians, healthcare professionals, or agents thereof, e.g., generally represented as caregiver(s) or clinician(s) 338.

Example patient(s) 302, each having a suitable implantable device 303, may be provided with a variety of corresponding external devices for controlling, programming, otherwise (re)configuring the functionality of respective implantable medical device(s) 303, as is known in the art. Such external devices associated with patient(s) 302 are referred to herein as patient devices 304 and may include a variety of user equipment (UE) devices, tethered or untethered, that may be configured to engage in remote care therapy sessions. By way of example, patient devices 304 may include smartphones, tablets or phablets, laptops/desktops, handheld/palmtop computers, wearable devices such as smart glasses and smart watches, personal digital assistant (PDA) devices, smart digital assistant devices, etc., any of which may operate in association with one or more virtual assistants, smart home/office appliances, smart TVs, virtual reality (VR), mixed reality (MR) or augmented reality (AR) devices, and the like, which are generally exemplified by wearable device(s) 306, smartphone(s) 308, tablet(s)/phablet(s) 310 and computer(s) 312. As such, patient devices 304 may include various types of communications circuitry or interfaces to effectuate wired or wireless communications, short-range and long-range radio frequency (RF) communications, magnetic field communications, BLUETOOTH communications, etc., using any combination of technologies, protocols, and the like, with external networked elements and/or respective implantable medical devices 303 corresponding to patient(s) 302.

With respect to networked communications, patient devices 304 may be configured, independently or in association with one or more digital/virtual assistants, smart home/premises appliances and/or home networks, to effectuate mobile communications using technologies such as Global System for Mobile Communications (GSM) radio access network (GRAN) technology, Enhanced Data Rates for Global System for Mobile Communications (GSM) Evolution (EDGE) network (GERAN) technology, 4G Long Term Evolution (LTE) technology, Fixed Wireless technology, 5th Generation Partnership Project (5GPP or 5G) technology, Integrated Digital Enhanced Network (IDEN) technology, WiMAX technology, various flavors of Code Division Multiple Access (CDMA) technology, heterogeneous access network technology, Universal Mobile Telecommunications System (UMTS) technology, Universal Terrestrial Radio Access Network (UTRAN) technology, All-IP Next Generation Network (NGN) technology, as well as technologies based on various flavors of IEEE 802.11 protocols (e.g., WiFi), and other access point (AP)-based technologies and microcell-based technologies such as femtocells, picocells, etc. Further, some embodiments of patient devices 304 may also include interface circuitry for effectuating network connectivity via satellite communications. Where tethered UE devices are provided as patient devices 304, networked communications may also involve broadband edge network infrastructures based on various flavors of Digital Subscriber Line (DSL) architectures and/or Data Over Cable Service Interface Specification (DOCSIS)-compliant Cable Modem Termination System (CMTS) network architectures (e.g., involving hybrid fiber-coaxial (HFC) physical connectivity). Accordingly, by way of illustration, an edge/access network portion 319A is exemplified with elements such as WiFi/AP node(s) 316-1, macro/microcell node(s) 316-2 and 316-3 (e.g., including micro remote radio units or RRUs, base stations, eNB nodes, etc.) and DSL/CMTS node(s) 316-4.

Similarly, clinicians 338 may be provided with a variety of external devices for controlling, programming, otherwise (re)configuring or providing therapy operations with respect to one or more patients 302 mediated via respective implantable medical device(s) 303, in a local therapy session and/or remote therapy session, depending on implementation and use case scenarios. External devices associated with clinicians 338, referred to herein as clinician devices 330, may include a variety of UE devices, tethered or untethered, similar to patient devices 304, which may be configured to engage in remote care therapy sessions as will be set forth in detail further below. Clinician devices 330 may therefore also include devices (which may operate in association with one or more virtual assistants, smart home/office appliances, VRAR virtual reality (VR) or augmented reality (AR) devices, and the like), generally exemplified by wearable device(s) 331, smartphone(s) 332, tablet(s)/phablet(s) 334 and computer(s) 336. Further, example clinician devices 330 may also include various types of network communications circuitry or interfaces similar to that of patient device 304, which may be configured to operate with a broad range of technologies as set forth above. Accordingly, an edge/access network portion 319B is exemplified as having elements such as WiFi/AP node(s) 328-1, macro/microcell node(s) 328-2 and 328-3 (e.g., including micro remote radio units or RRUs, base stations, eNB nodes, etc.) and DSL/CMTS node(s) 328-4. It should therefore be appreciated that edge/access network portions 319A, 319B may include all or any subset of wireless communication means, technologies and protocols for effectuating data communications with respect to an example embodiment of the systems and methods described herein.

In one arrangement, a plurality of network elements or nodes may be provided for facilitating a remote care therapy service involving one or more clinicians 338 and one or more patients 302, wherein such elements are hosted or otherwise operated by various stakeholders in a service deployment scenario depending on implementation (e.g., including one or more public clouds, private clouds, or any combination thereof). In one embodiment, a remote care session management node 320 is provided, and may be disposed as a cloud-based element coupled to network 318, that is operative in association with a secure communications credentials management node 322 and a device management node 324, to effectuate a trust-based communications overlay/tunneled infrastructure in network environment 300 whereby a clinician may advantageously engage in a remote care therapy session with a patient.

In the embodiments described herein, implantable medical device 303 may be any suitable medical device. For example, implantable medical device may be a neurostimulation device that generates electrical pulses and delivers the pulses to nervous tissue of a patient to treat a variety of disorders.

In some embodiments, the neurostimulation system is adapted to provided neural activity inhibiting or blocking stimulation as discussed herein.

In many applications of neurostimulation, the pulses applied to neural tissue of the patient evoke compound action potentials (ECAPs) (also referred to a neuronal activation). For example, traditional spinal cord stimulation (SCS) with tonic stimulation waveform activates the dorsal column fibers. The activated dorsal column fibers in turn activate the inhibitory interneurons and the activated inhibitory interneurons inhibit the dorsal horn projection of the spinal cord.

The electrical current of pulses causes depolarization of the stimulated neural cells. ECAPs represent the synchronous firing of a population of electrically stimulated nerve fibers. Measurement of ECAPs has been utilized as a means of directly assessing the level of fiber recruitment in the dorsal columns of the spinal cord for neurostimulation. ECAP sensing may also be applied for other neural targets in a similar manner. ECAPs are signals elicited by electrical stimulation and recorded near a bundle of fibers. In particular, ECAPs usually arrive less than one millisecond (<1 ms) after a corresponding stimulation pulse and last in the range of approximately one half to one millisecond (0.5-1 ms). ECAPs may be measured and analyzed, for example, to evaluate and/or control the comfort and efficacy of a SCS treatment regimen (see e.g., US Patent Publication Nos. 2020/0282208, 2011/0018448; and 2020/0391031 the disclosures of which are incorporated herein by reference).

However, direct inhibition of neurons may be useful in different neuromodulation applications as opposed to direct activation of neural tissue. For example, maladaptation of voltage gated calcium channels (VGCC) is believed to be related with neuropathic pain and VGCC blockers have been shown to be effective in mitigating chronic pain. The VGCC is normally closed at resting potential. It opens when cells are depolarized. Therefore, maintaining the cells in a resting state or hyperpolarized state would keep the VGCC closed and prevent cascading effect of calcium influx. Anodic direct current (DC) stimulation can keep the cells hyperpolarized. However, DC stimulation cannot be used for implantable electrodes due to safety concerns.

Some embodiments provide a novel stimulation waveform that hyperpolarize neurons which can be used to mitigate pain by directly suppressing the nociceptive projection neurons in the superficial lamina of spinal dorsal horn. Embodiments of the inhibiting stimulation waveform keep a neuron population hyperpolarized to reduce the neuronal excitability. In some embodiments, the stimulation waveform is composed of a sequence of subthreshold anodic pulses with gradual increase of the stimulation amplitude and/or pulse width followed by cathodic discharge phase with intermittent time allocated for discharge to prevent cell activation during discharge.

Utilizing the knowledge of the time constants of different voltage gated ion channels, a stimulation waveform may be constructed that avoids action potential generation but induces subthreshold transmembrane potential change to make the neurons less excitable.

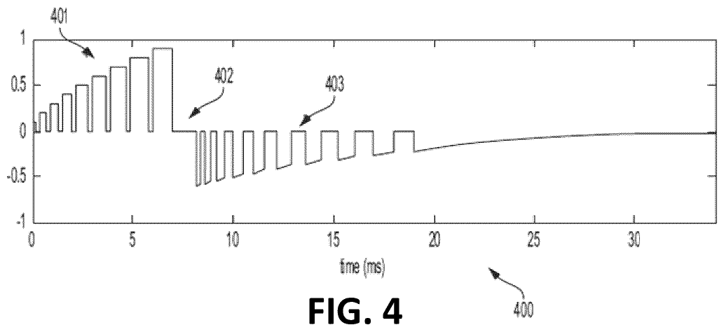

In some embodiments, inhibiting waveform 400 (shown in FIG. 4) includes the following elements in a sequential order: 1) a charge injection phase; 2) an optional time delay; and 3) a passive discharge phase.

In some embodiments, the charge injection phase 401 may consist of a sequence of anodic pulses with gradually increasing pulse amplitude and/or pulse width with a gap between the pulses. The stimulation amplitude of each pulse is set to below the threshold amplitude to avoid generating action potentials. A sequence of the sub-threshold anodic pulses 401 hyperpolarizes a cell. In another implementation, there can be optional active/passive discharge between the pulses during the inter-pulse interval of the charge injection phase 401.

In some embodiments, after charge injection and before discharge there can be optional gap 402 to extend the hyperpolarized membrane status.

In some embodiments, discharge phase 403 is provided. During discharge phase 403, passive discharge is used to recover the electric charge stored in a DC blocking capacitor and double layer capacitance. In order to prevent action potential (AP) generation from anodic breaking, the discharge occurs gradually with increased time allocated for discharge. The amplitude and pulse width of cathodic pulses occurring during the discharge phase are not high enough to generate AP.

In some embodiments, in order to minimize electrode corrosion or tissue damage due to irreversible Faradaic reaction at the electrode-tissue interface, the stimulation amplitude may be maintained at a low level (e.g., 100 uC/cm² for total anodic pulses in spinal cord stimulation applications).

In some embodiments, IPGs (such as IPG 150) are adapted to provide the inhibiting waveform to neural tissue of a patient. Additionally, external patient and/or clinician devices (such as device 160) are adapted to program and/or control IPG 150 to apply the inhibiting waveform as directed by the patient and/or clinician. In operation, IPG 150 repeatedly generate inhibiting waveform 400 in succession and applies waveform 400 to tissue of the patient using one or more electrodes of an implantable lead. In some embodiments, the one or more electrodes are implanted within the epidural space of the patient to inhibit dorsal fibers of the spinal cord. This inhibition may mitigate pain by directly suppressing nociceptive neurons in the superficial lamina of the spinal dorsal horn.

In some embodiments, stimulation waveform parameters are selected to optimize waveform 400 for a patient. For example, the pulse amplitude, pulse width, pulse gaps/delays are selected to inhibit neural activity of the patient. A clinician may manually program one or more of the respective stimulation parameters according to some embodiments. Additionally or alternative, IPG 150 may conduct sensing operations while stimulation is provided according to a range of stimulation parameters and, in response thereto, select suitable parameters for waveform 400 for the patient.

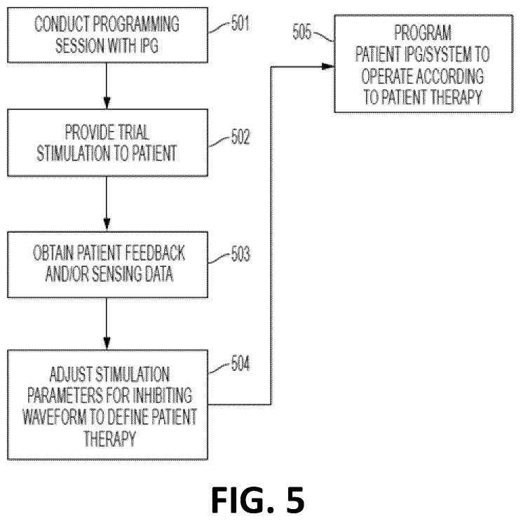

FIG. 5 depicts defining a neurostimulation therapy for a patient according to some embodiments. In 501, a programming session is conducted with the patient IPG. The programming session may be an in-person programming session in a conventional clinic setting or may be a remote programming session. In 502, trial stimulation is provided to the patient. The trial stimulation provides the inhibiting waveform to the patient across a range of stimulation parameters to identify optimal stimulation parameters for the patient.

In 503, patient feedback and/or sensing data is obtained. In 503, the feedback and/or sensing data may confirm that the inhibiting waveform is sub-threshold and is not generating action potentials. In some embodiments, stimulation pulses according to the inhibiting waveform may be provided on a first electrode or set of electrodes and sensing may be conducted on a second electrode or set of electrodes. The sensing on the second electrode(s) may verify that the inhibiting waveform is inhibiting neural activity in an expected manner. For example, initial sensing may occur before stimulation is provided to establish a baseline signal for comparison. Thereafter, while the inhibiting waveform is applied, ECAP or other suitable sensing may be conducted to compare against the baseline signal to verify that the inhibiting waveform has reduced and/or eliminated neural activity.

Based on the patient feedback and/or sensed data, the stimulation parameters are adjusted in 504 for the inhibiting waveform to define the patient's therapy. In 505, the patient IPG/system is programmed to operate according to the defined patient therapy.

Also, in some embodiments, the sensing operations may be applied by the IPG to adjust therapy parameters in a closed-loop manner.

Traditional tonic spinal cord stimulation (SCS) activates the dorsal column axons, which in turn reduces the hyperactivity of the dorsal horn projection neurons. Alternatively, paresthesia free waveforms may modulate the transmembrane potential of the projection neurons without the dorsal column fiber activation. In some embodiments disclosed herein, novel stimulation waveforms are disclosed and may be used to deliver neurostimulation therapy to patients to treat a medical condition of the patient, such as pain, by suppressing the spontaneous activity of the projection neurons of spinal dorsal horn.

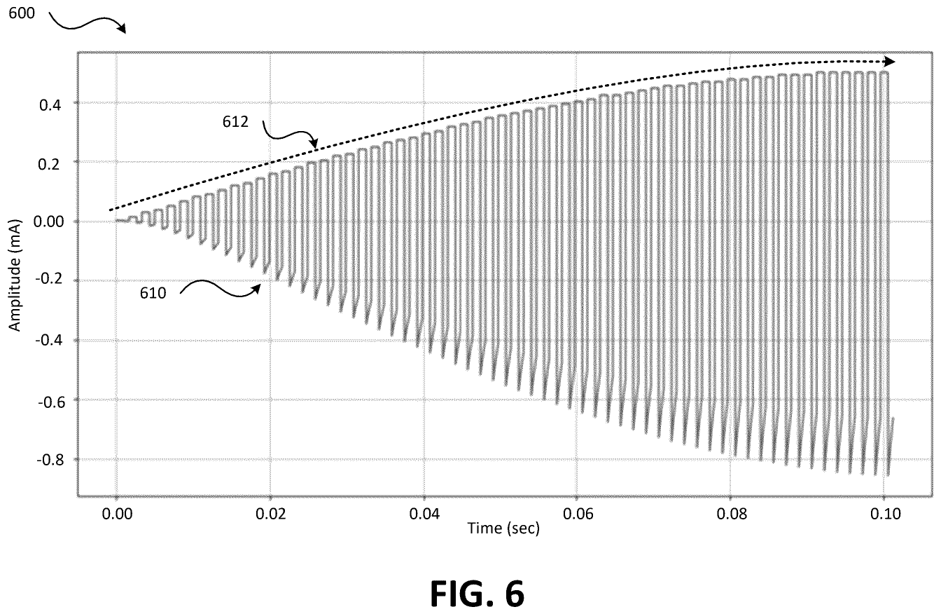

Novel stimulation waveforms consist of a train of pulses with long pulse widths and short inter-pulse intervals (IPIs). After a train of pulses, another train of pulses having an opposite phase can optionally follow the train of pulses. A pulse amplitude and pulse width of each pulse may be maintained below a threshold, such as a threshold to generate a sensation of paresthesia in the patient. In some embodiments, waveforms may have a pulse train of constant or varying pulse amplitude. The pulse width (PW) may be set to be long (0.5 ms≤PW≤2 ms) and the IPI may be set to be relatively short (0.1 ms≤IPI≤1 ms). During IPI, charge accumulated in the DC blocking capacitor is passively discharged. Non-limiting examples of stimulation waveforms in accordance with the concepts described herein are described with reference to FIGS. 6-11.

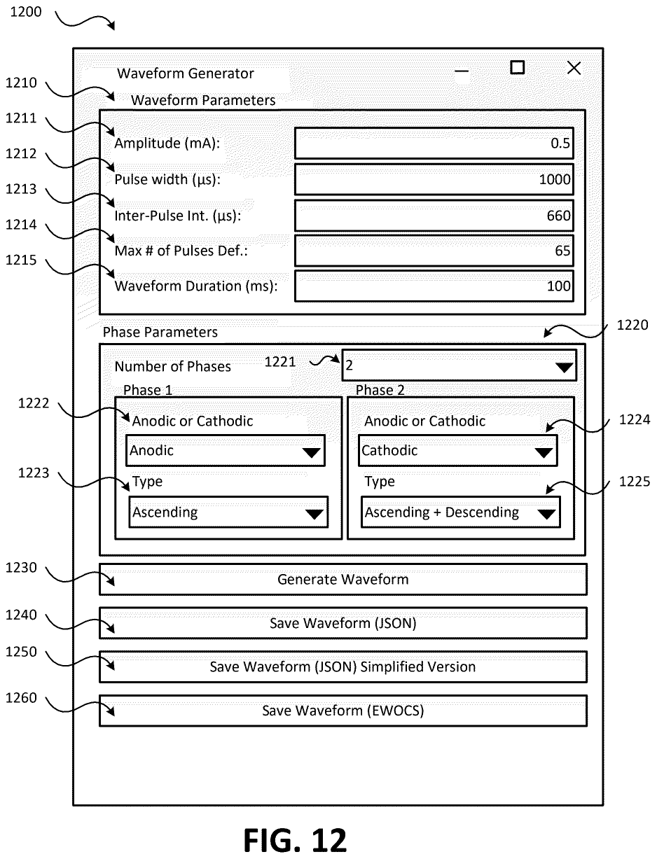

A waveform program running on a clinician programmer device (e.g., one of clinician devices 330 of FIG. 3) may be utilized to generate a pulse definition and interval definition. In an aspect, the pulse definition and interval definition may be generated within a limited memory size of an IPG. For example, the program memory size of an IPG may be a few kilobytes (kB) to tens of kB (e.g., 20 kB, 30 kB, 50 kB, between 50-100 kB). It is noted that the program memory size of the IPG may vary depending on the memory configuration of the IPG and that some IPGs may have more memory space available for programs than others. Further, the ability to store programs (e.g., stimulation programs having pulse definitions and interval definitions defined in accordance with the concepts described herein) may be dependent on the number of programs stored in the program memory (e.g., storing more or larger programs may reduce the available program memory space for new programs). Thus, even for IPGs with larger memories, available memory space for storing programs may become limited over time. Aspects of a user interface (UI) that may be utilized to configure and generate waveforms according to some embodiments are described in more detail below with reference to FIG. 12. Non-limiting examples of waveforms generated according to some embodiments are described in more detail below.

In FIGS. 6-13 below, example stimulation waveforms and methods of generating them are described. It is noted that the various stimulation waveforms may include: a) repetition of a burst of electrical pulses of one or two phases of selectable anodic or cathodic polarities based on respective ones of the inputs; (b) respective pulses of the one or two phases are separated by a time gap; (c) the amplitude of respective pulses within the one or two phases vary in manner that is increasing, decreasing, or combination thereof based on respective input parameters configured for the stimulation waveform; and (d) the electrical pulses of the one or two phases are charge limited such that the stimulation waveform is sub-threshold. Electrical pulses of a stimulation waveform in accordance with the concepts described herein may be used to treat pain of a patient (e.g., via spinal cord stimulation (SCS) and the like), a movement disorder of the patient, or other medical conditions. In an aspect, the sub-threshold electrical pulses may not create paresthesia.

Referring to FIG. 6, a diagram illustrating an example waveform exhibiting a gradual increase of pulse amplitude in accordance with some embodiments is shown as a waveform 600. The waveform 600 of FIG. 6 includes a train of pulses 610 having a pulse amplitude (y-axis) that increases over a time interval (x-axis). In the example shown in FIG. 6, the pulse amplitude increases from about 0.00 mA at 0.00 seconds(s) to approximately 0.5 mA at about 0.10 seconds(s), as indicated by arrow 612.

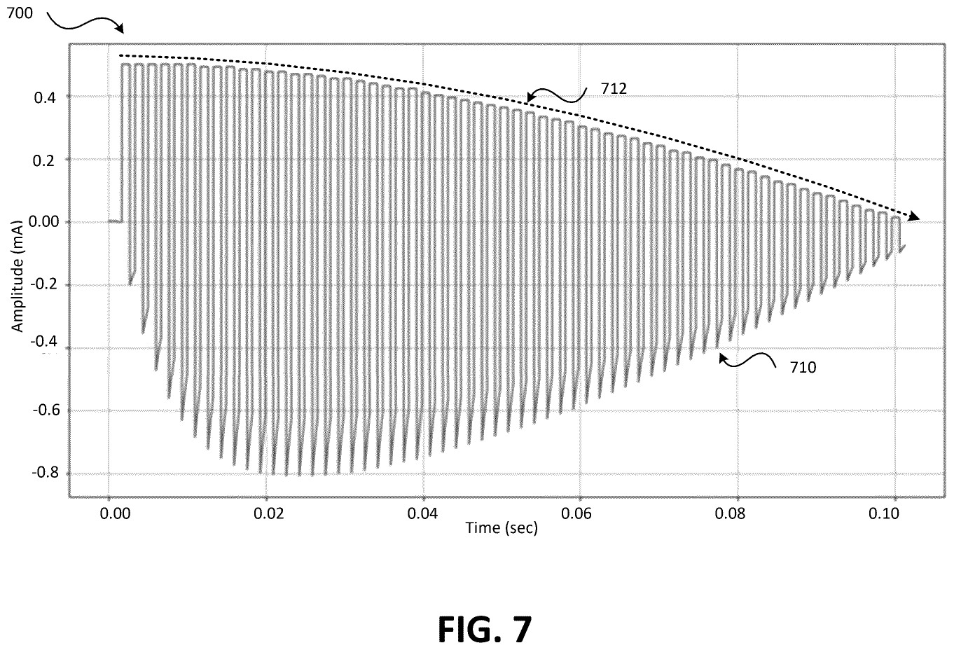

Referring to FIG. 7, a diagram illustrating an example waveform exhibiting a gradual decrease of pulse amplitude in accordance with some embodiments is shown as a waveform 700. The waveform 700 includes a train of pulses 710 having a pulse amplitude (y-axis) that decreases over a time interval (x-axis). In the example shown in FIG. 7, the pulse amplitude decreases from about 0.5 mA at 0.00 s to approximately 0.00 mA at about 0.10 s, as indicated by arrow 712.

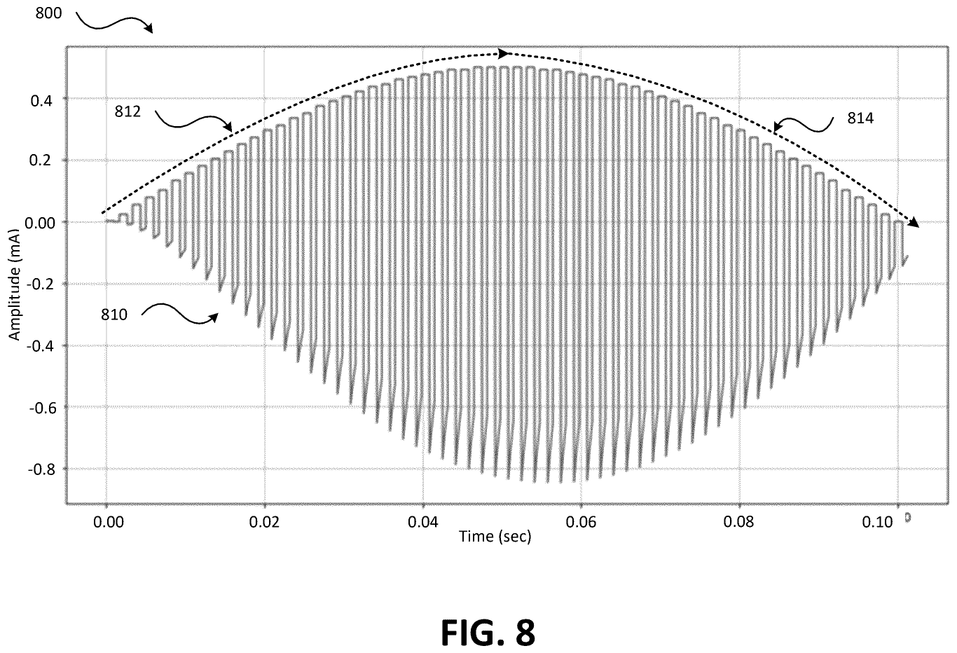

Referring to FIG. 8, a diagram illustrating an example waveform exhibiting a gradual increase of pulse amplitude followed by gradual decrease of the pulse amplitude in accordance with some embodiments is shown as a waveform 800. The waveform 800 includes a train of pulses 810 having a pulse amplitude (y-axis) that increases and decreases over a time interval (x-axis). In the non-limiting example shown in FIG. 8, the pulse amplitude of the train of pulses 810 increases from about 0.00 mA to about 0.5 mA over a first portion of the time interval (e.g., from 0.00 s to about 0.05 s), as indicated by arrow 812, and then decreases from about 0.5 mA to about 0.00 mA over the time interval from 0.05 s to about 0.10 s, as indicated by arrow 814.

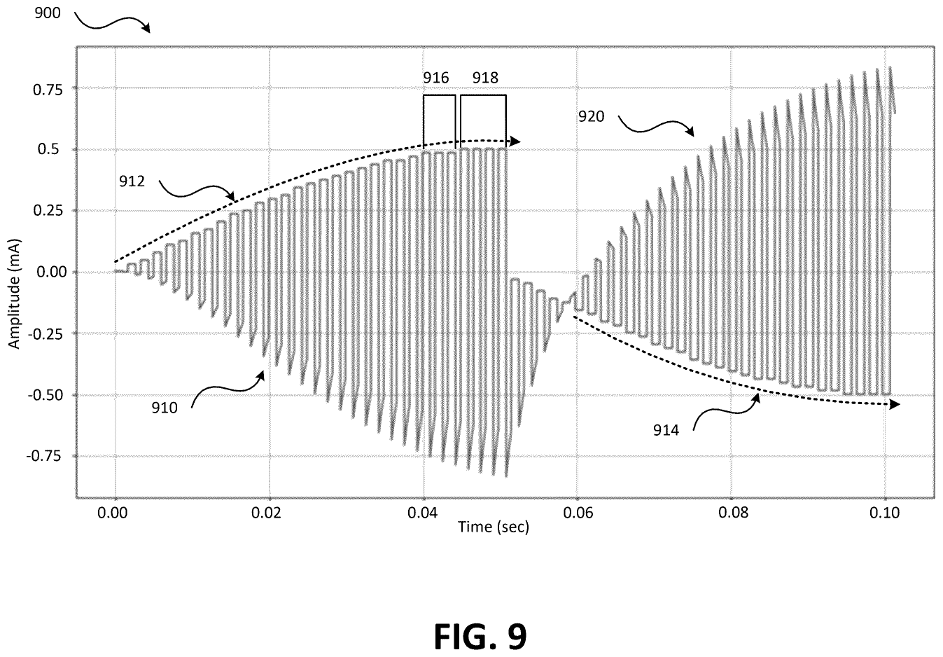

Referring to FIG. 9, a diagram illustrating an example waveform having an anodic pulse train with gradual increase of the pulse amplitude followed by a cathodic pulse train with gradual increase of the pulse amplitude in accordance with some embodiments is shown as a waveform 900. The waveform 900 includes an anodic train of pulses 910 having a pulse amplitude (y-axis) that gradually increases followed by a cathodic train of pulses 920 having a pulse amplitude (y-axis) that gradually decreases. In the non-limiting example shown in FIG. 9, the pulse amplitude of the anodic train of pulses 910 increases from about 0.00 mA to about 0.5 mA over a first portion of the time interval (e.g., from 0.00 s to about 0.05 s), as indicated by arrow 912, and the pulse amplitude of the cathodic train of pulses decreases from about 0.00 mA to about −0.5 mA over the time interval from 0.05 s to about 0.10 s, as indicated by arrow 914. The anodic pulse train may be utilized to hyperpolarize a transmembrane potential of the neurons at a target stimulation site (e.g., neurons underneath the anode(s)) and supress spontaneous activity of the neurons. A gradual increase of the amplitude may be utilized to prevent initial onset of action potential generation of the neurons underneath the cathode(s). A Time intervals for the stimulation can be expanded beyond 0.1 s (e.g. 0.2 s, 0.3 s, 0.3-0.5 s, 0.5-1.0 s, etc.). However, as noted above, available program memory may limit the ability to extend the duration of the stimulation.

In some embodiments, the pulse amplitude of a subset of pulses in a train of pulses may be approximately the same and the pulse amplitude of adjacent subsets of pulses may be different with respect to each other. For example, in the train of pulses 910 of FIG. 9 a subset of pulses 916 may have approximately a same pulse amplitude and a next subset of pulses 918 may have approximately a same pulse amplitude, but the pulse amplitude of the subsets of pulses 916 and 918 may be different (e.g., a pulse amplitude of the subsets of pulses 916 may be less than a pulse amplitude of the subsets of pulses 918). It is noted that adjacent subsets of pulses may also have decreasing differences in pulse amplitude instead of increases in pulse amplitude in some embodiments. Additionally, it is noted that invidiual pulses within a subset of pulses may have slight or small variation in pulse amplitude (e.g., increasing or decreasing variations), but the variation in pulse amplitude between pulses within a subset may be smaller (i.e., less change between adjacent pulses) compared to a pulse amplitude of pulses in a subset immediately before or after the subset (e.g., variation between sequential increases in pulses of the subset 918 may be relative to a difference between the pulse amplitude of a last pulse of the subset 916 and a first pulse of the subset 918).

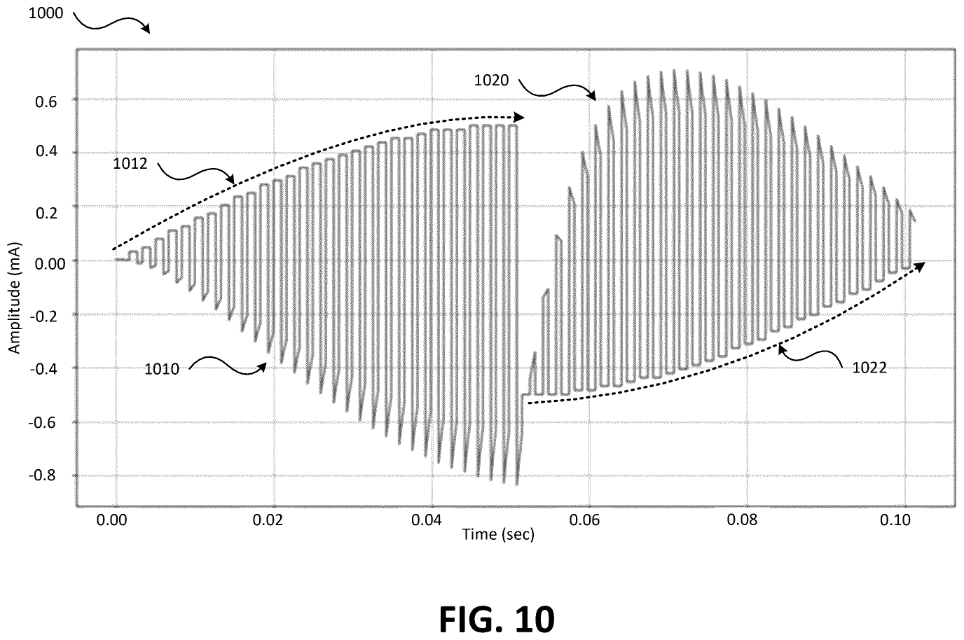

Referring to FIG. 10, a diagram illustrating an example waveform having an anodic pulse train with gradual increase of the pulse amplitude followed by a cathodic pulse train with gradual decrease of the pulse amplitude in accordance with some embodiments is shown as a waveform 1000. The waveform 1000 includes an anodic train of pulses 1010 having a pulse amplitude (y-axis) that gradually increases followed by a cathodic train of pulses 1020 having a pulse amplitude (y-axis) that gradually increases. In the non-limiting example shown in FIG. 10, the pulse amplitude of the anodic train of pulses 1010 increases from about 0.00 mA to about 0.5 mA over a first portion of the time interval (e.g., from 0.00 s to about 0.05 s), as indicated by arrow 1012, and the pulse amplitude of the cathodic train of pulses 1020 increases from about −0.50 mA to about −0.00 mA over the time interval from 0.05 s to about 0.10 s, as indicated by arrow 1022.

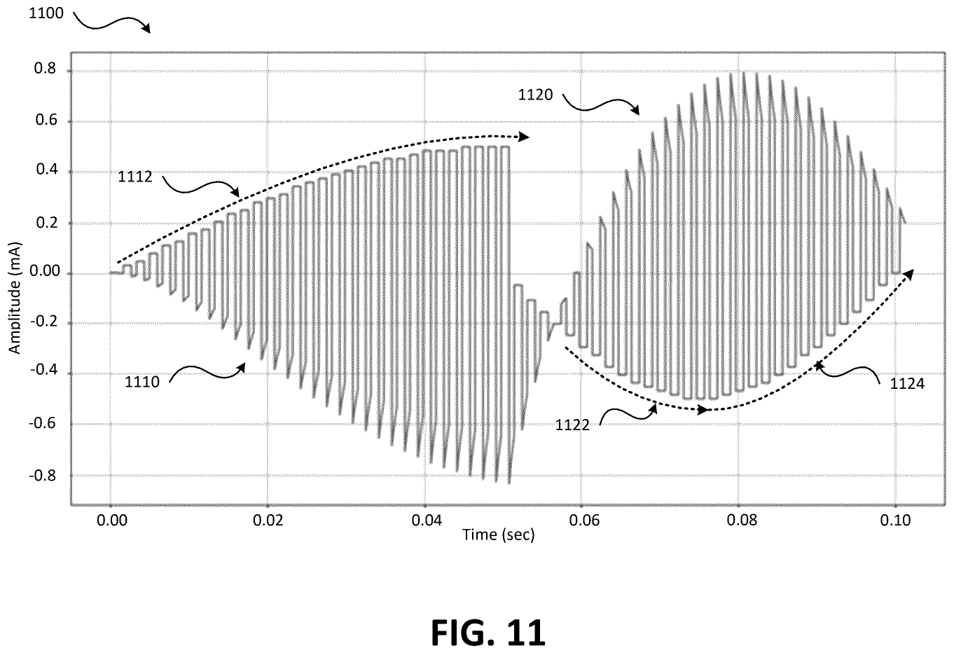

Referring to FIG. 11, a diagram illustrating an example waveform having an anodic pulse train with gradual increase of the pulse amplitude followed by a cathodic pulse train with a gradual decrease of the pulse amplitude followed by a gradual increase in pulse amplitude in accordance with some embodiments is shown as a waveform 1100. The waveform 1100 includes an anodic train of pulses 1110 having a pulse amplitude (y-axis) that gradually increases followed by a cathodic train of pulses 1120 having a pulse amplitude (y-axis) that gradually decreases and then increases. In the non-limiting example shown in FIG. 11, the pulse amplitude of the anodic train of pulses 1110 increases from about 0.00 mA to about 0.5 mA over a first portion of the time interval (e.g., from 0.00 s to about 0.5 s), as indicated by arrow 1112, and the pulse amplitude of the cathodic train of pulses decreases from about −0.25 mA to about −0.50 mA over a portion of the time interval (e.g., from 0.055 s to about 0.075 s), as indicated by arrow 1122 and then increases from about −0.50 mA to about 0.00 mA over a portion of the time interval (e.g., from 0.075 s to about 0.10 s).

In some embodiments, a rate of the decrease or increase in pulse amplitude for a train of pulses may be uniform over the duration of the pulse train such that the change of the pulse amplitude provides a uniform rate of change (e.g., increase or decrease) of time. For example, in FIG. 11 the rate of increase for the train of pulses 1110 increase relatively uniformly over time, as indicated by arrow 1112 and the rate of decrease in pulse amplitude for the train of pulses 1120 is relatively uniform over time, as indicated by arrow 1122. In some embodiments, the rate of the decrease or increase in pulse amplitude for a train of pulses may be non-uniform over the duration of the pulse train such that the change of the pulse amplitude has a non-uniform rate of change (e.g., increase or decrease) of time. For example, in FIG. 11 the rate of increase for the train of pulses 1120 increases more rapidly over time, as indicated by arrow 1124. It is noted that the variations in the characteristics of the pulse trains (e.g., rates of change, increases and/or decreases in amplitude, etc.) may have different impacts on the therapeutic effect on the patient. Accordingly, such variations may be used to control therapeutic properties of the stimulation on a given patient. In an aspect, the aforementioned variations may be tested on an individual patient to determine what, if any, impact the changes in characteristics of the pulse trains have on the symptoms being treated, and a final configuration of a stimulation waveform to provide therapy to the patient may be determined based on the results of the trial stimulation.

As can be appreciated from the description of FIGS. 6-11 above, waveforms generated according to some embodiments may include anodic waveforms, cathodic waveforms, or waveforms including pulse trains associated with anodic and cathodic trains of pulses. In some embodiments, a first pulse train can be anodic and a second pulse train can be cathodic. In some embodiments, a first pulse train can be cathodic and a second pulse train can be anodic. In some embodiments, more than one pulse train may be configured and various ones of the more than one pulse train can be anodic or cathodic and may have changing pulse amplitudes. A pulse amplitude of the train(s) of pulses may increase over time, decrease over time, increase and then decrease over time, or decrease and then incease over time. Where a waveform includes both anodic and cathodic trains of pulses, the change in pulse amplitude of both the anodic and cathodic tarins of pulses may change over time as described above (e.g., increase, decrease, increase then decrease, or decrease and then increase). It is noted that the rate of increase or decrease of the train of pulses may be gradually increasing or decreasing, or may increase or decrease rapidly. The change in pulse amplitude can be sinusoidal, linear, exponential, and the like. In some embodiments, there may be virtually no gap between different pulse trains when a waveform includes multiple trains of pulses (e.g., pulse train 1 may end at 0.05 s and pulse train 2 may start between 0.051 s and 0.055 s). In some embodiments, there may be some gap between different pulse trains when a waveform includes multiple trains of pulses (e.g., a gap of 1 ms (0.01 s) to 1 or more minutes). In some embodiments, a duration of each train of pulses may be between 10 ms to 1000 ms (0.01 s to 1 s). Unlike the wvaeform in FIG. 4, which utilized an increasing PW over a train of pulses, the PW of the pulses shown in FIGS. 6-11 may be maintained as constant. In an aspect, waveforms generated in accordance with some embodiments may begin with plurality of anodic pulses that are charge limited to be sub-threshold (e.g., below a perception threshold for paresthesia). The anodic pulses may hyperpolarize nociceptive projection neurons of a dorsal horn of a spine of the patient, thereby keeping the projection neurons from activating. The anodic pulses may followed by a discharge phase of intermittent time periods to discharge charge build up from the anodic pulses. In an aspect, respective pulses of the one or two phases are separated by a time gap. In an aspect, the time gap separating respective pulses of the stimulation wacveform may be uniform for a single phase but may vary for different phases (e.g., a first phase and second phase may have different time gaps). In an additional or alternative aspect, the time gap separating respective pulses of the stimulation wacveform may be uniform for all phases (e.g., a first phase and second phase may have the same time gap).

Referring to FIG. 12, a block diagram illustrating an example user interface (UI) for generating stimulation waveforms in accordance with some embodiments is shown as a UI 1200. In an aspect, the UI 1200 may be presented at a computing device, such as a clinician programmer, or another device suitable for configuring waveforms used for treatment of one or more patient conditions in accordance with the concepts described herein. For example, the UI 1200 may be utilized to generate the waveforms described above and illustrated with refrence to FIGS. 6-11.

As shown in FIG. 12, the UI 1200 includes a waveform parameters region 1210, a phase parameters region 1220, and interative elements 1230-1260 for generating and saving waveforms designed in accordance with some embodiments. The waveform parameters region 1210 includes interactive controls 1211-1215 for configuring certain waveform parameters of a stimulation waveform. In particular, interactive control 1211 provides a data field for configuring one or more amplitude parameters (specified in milliamperes (mA)). For example, FIG. 12 illustrates an input value for an amplitude of 0.5 mA. Interactive control 1212 provides a data field for configuring one or more pulse width parameters (specified in microseconds (μs)). In FIG. 12 interactive elements 1212 is shown to include an input value for the pulse width parameter of 1000 μs. In an aspect, values input to interactive element 1212 may be constrained to specific values, such as values that are multiples of 20 (e.g., 20, 40, 60, . . . , 1000, and so on). If an input value is not specified within the constrained parameter values, the interactive element 1212 may automatically round the value to a nearest constrained value (e.g., round 991 to 1000 or round 988 to 980). Additionally or alternatively, the UI 1200 may be configured to provide an alert to indicate an input value does not satisfy one or more contraints imposed on the parameter values.

Interactive control 1213 provides a data field for configuring one or more inter-pulse interval parameters (specified in microseconds (μs)). In FIG. 12 interactive elements 1213 is shown to include an input value for the inter-pulse interval parameter of 660 μs. In an aspect, values input to interactive element 1213 may be constrained to specific values, such as values that are multiples of 20 (e.g., 20, 40, 60, . . . , 1000, and so on). If an input value is not specified within the constrained parameter values, the interactive element 1213 may automatically round the value to a nearest constrained value (e.g., round 651 to 660 or round 649 to 640). Additionally or alternatively, the UI 1200 may be configured to provide an alert to indicate an input value does not satisfy one or more contraints imposed on the parameter values. Interactive control 1214 provides a data field for configuring a definition for a maximum number of pulses parameter. In FIG. 12 interactive elements 1214 is shown to include an input value for the definition for a maximum number of pulses parameter of 65. Interactive element 1215 provides a data field for configuring a waveform duration (specified in milliseconds (ms)). In FIG. 12 interactive element 1215 is shown to include an input value for the waveform duration parameter of 100 ms.

The phase parameters region 1220 includes interactive controls 1221-1225 for configuring certain phase parameters of a stimulation waveform according to some embodiments. In particular interactive control 1221 enables a clinician to configure a number of phases of the stimulation waveform. In the non-limiting example of FIG. 12, the number of phases is set to 2 via a dropdown menu. Interactive elements 1222 and 1223 enable a clinician to configure parameters of a first phase of the stimulation waveform, and interactive elements 1224 and 1225 enable the clinician to configure parameters of a second phase of the stimulation waveform. For example, interactive elements 1222, 1224 enable the clinician configure the first and second phases to provide anodic or cathodic stimulation for the first and second phases, respectively, and interactive elements 1223, 1225 enable the clinician configure the first and second phases to provide ascending, descending, ascending and descending, or descending and ascending types of stimulation for the first and second phases, respectively. It is noted that different phases of the stimulation waveform may have a same configuration or different configurations for one or more of the parameters associated with the phase parameters region 1220. For example, one or more phases may be configured to be anodic and one or more other phases may be configured to be cathodic (e.g., as in FIGS. 9-11), or all phases may be configured to be anodic or cathodic. Additionally, different phases may have the same or different phase types (e.g., ascending, descending, ascending and descending, or descending and ascending).

Interactive elements 1230-1260 of the UI 1200 may be buttons or other interactive UI control elements that may be activated by interaction (e.g., clicking or tapping on a touchscreen) to perform various tasks. For example, activation of the interactice element 1230 may generate a stimulation waveform according to the parameters specified in the UI 1200 (e.g., based on values configured in the waveform parameters region 1210 and the phase parameters region 1220). Activation of interactive element 1240 may cause programming data corresponding to the waveform to be saved in a first format (e.g., a JavaScript Object Notation (JSON) format) and activation of interactive element 1250 may cause programming data corresponding to the waveform to be saved in a second format (e.g., a JSON simplified format) that is different from the first format. Activation of the interactive element 1260 may cause programming data corresponding to the waveform to be saved in a third format (e.g., an EWOCS format), which may be different from the first and second formats. Once saved, the programming data may be transmitted to an IPG as programming data and the programming data corresponding to the waveform may be stored in a memory of the IPG. Additionally, external patient and/or clinician devices (such as device 160) are adapted to program and/or control IPG 150 to apply the inhibiting waveform as directed by the patient and/or clinician. In operation, IPG 150 repeatedly generates inhibiting waveforms (e.g., waveforms similar to those described with reference to FIGS. 6-11) in succession and applies the waveform to tissue of the patient using one or more electrodes of an implantable lead. In some embodiments, the one or more electrodes are implanted within the epidural space of the patient to inhibit activity of neural tissue of the spinal cord of the patient, such as projection neurons of the spinal dorsla horn. This inhibition may mitigate pain by directly suppressing nociceptive neurons in the superficial lamina of the spinal dorsal horn.

As can be appreciated from the description above, the UI 1200 provides functionality for configuring stimuilation waveforms. In some embodiments, a lossy coding of pulse amplitude may be adopted with optimization of the pulse definition when the number of unique pulse amplitudes in the waveform exceeds the input maximum number of pulses definition. In some embodiments, the waveform duration may be truncated if the input waveform duration produces a program that is longer than an amount of memory available in an IPG (e.g., the IPG 150), thereby enabling the waveforms to be utilized in IPGs having limited memory available. In an additional or alternative aspect, the waveform program may be split into multiple programs that may be loaded into limited memory of the IPG sequentially, thereby overcoming memory limitations in the IPG. It is noted that the various features of the UI 1200 and the interactice elements described above have been provided for purposes of illustration, rather than by way of limitation and that UIs providing functionality to support generation of waveforms and IPG programming data for generation of waveforms in accordane with the concepts described herein may provide additional configurable parameters, utilize other types of interactice elements, and other functionality consistent with the concepts disclosed herein. Stimualtion delivered to patients in accordance with programming data generated using the UI 1200 and the concepts described herein may be used to treat patient pain or other medical conditions. For example, stimulation of neural tissue using a waveform configured in accordance with some embodiments may suppress spontaneous activity of projection neurons of the spinal dorsal horn, thereby mitigating pain of the patient. To illustration, for suppression of projection neuronal activity of the dorsal horn, more lateral and cordal lead placement over the dorsal horn may be suitable as compared to conventional lead placement.

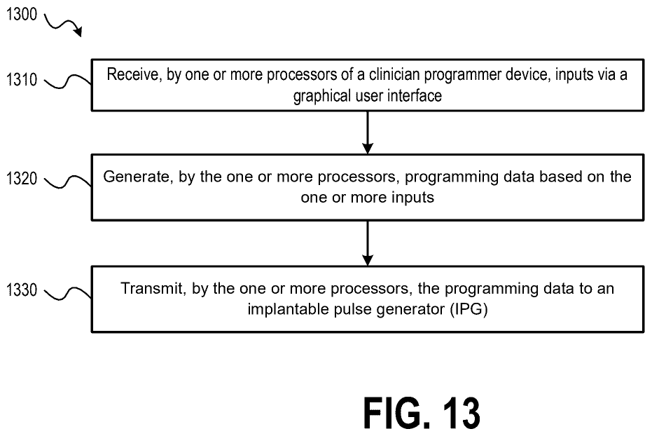

Referring to FIG. 13, a flow diagram of an example method for providing a neurostimulation therapy to a patient in accordance with some embodiments is shown as a method 1300. In some embodiments, the method 1300 may be performed by an IPG, such as the IPG 150 of FIG. 1. Steps of the method 1300 may be stored as instructions (e.g., programming data) in a memory of the IPG and may be executed to delivery neurostimulation therapy to the patient in accordance with embodiments of the present disclosure.

At step 1310, the method 1300 includes receiving, by one or more processors of a clinician programmer device, inputs via a graphical user interface. In an aspect, the inputs correspond to a set of parameters for configuring a stimulation waveform such that the waveform parameters and the phase parameters configure electrical properties of electrical pulses of the stimulation waveform and a shape of the stimulation waveform. For example, the stimulation waveform may comprise: (a) repetition of a burst of electrical pulses of one or two phases of selectable anodic or cathodic polarities based on respective ones of the inputs; (b) respective pulses of the one or two phases are separated by a time gap; (c) the amplitude of respective pulses within the one or two phases vary in manner that is increasing, decreasing, or combination thereof based on respective ones of the inputs; and (d) the electrical pulses of the one or two phases are charge limited such that the stimulation waveform is sub-threshold.

At step 1320, the method 1300 includes generating, by the one or more processors, programming data based on the one or more inputs. As described above, the programming data may be configured to control generation of a stimulation waveform based on the set of parameters. At step 1330, the method 1300 includes transmitting, by the one or more processors, the programming data to an implantable pulse generator (IPG). The IPG may utilize the programming data to generate electrical pulses according to the stimulation waveform defined in step 1310 and deliver the electrical pulses to tissue of a patient via one or more leads implanted in the patient to treat a medical condition of the patient (e.g., pain, a movement disorder, and the like).

In an aspect, the method 1300 may be performed during programming of the IPG. For example, a clinician may utilize a clinician programmer device and the UI 1200 to design one or more waveforms having anodic, cathodic, or anodic and cathodic pulse trains having uniform PW and increasing, decreasing, increasing and decreasing, or decreasing and increasing pulse amplitudes. Any number of pulse train phases (e.g., cathodic phases and anodic phases) may be defined and used to generate programming data that may be transmitted to the IPG to program the IPG to generate electrical pulses according to the waveform defined by the programming data. Once programmed into the IPG, the waveform may be used to generate electrical pulses that may be delivered to the patient to treat a medical condition of the patient. It is noted that the method 1300 may incorporate any of the concepts described above with reference to FIGS. 1-13.
Clause 1: A method of providing a neurostimulation therapy to a patient, comprising: receiving, by one or more processors of a clinician programmer device, inputs via a graphical user interface, the inputs corresponding to a set of parameters for configuring a stimulation waveform such that the waveform parameters and the phase parameters configure electrical properties of electrical pulses of the stimulation waveform and a shape of the stimulation waveform, wherein (a) the stimulation waveform comprises repetition of a burst of electrical pulses of one or two phases of selectable anodic or cathodic polarities based on respective ones of the inputs; (b) respective pulses of the one or two phases are separated by a time gap; (c) the amplitude of respective pulses within the one or two phases vary in manner that is increasing, decreasing, or combination thereof based on respective ones of the inputs; and (d) the electrical pulses of the one or two phases are charge limited such that the stimulation waveform is sub-threshold; generating, by the one or more processors, programming data based on the one or more inputs, wherein the programming data is configured to control generation of a stimulation waveform based on the set of parameters; and transmitting, by the one or more processors, the programming data to an implantable pulse generator (IPG).
Clause 2: The method of clause 1, wherien the waveform parameters comprise a pulse amplitude parameter defining maximum amplitude of the electrical pulses, a pulse width parameter defining a duration of each electrical pulse, an inter-pulse interval parameter defining a duration of time between each electrical pulse, a maximum number of pulses definition parameter defining a maximum number of pulses in the stimulation waveform, a waveform duration parameter defining a maximum duration of the stimulation waveform, or a combination thereof.
Clause 3: The method of clause 1 or 2, wherein the of electrical pulses are charge limited to be sub-threshold.
Clause 4. The method of any of clauses 1-3, wherien the waveform parameters comprise a number of phases parameter defining a number of phases of the stimualtion waveform, a phase type parameter to configure a phase type for each phase of the stimulation waveform, a phase mode parameter to configure a mode for each phase of the stimulation waveform, wherein the phase type is one of successively increasing charge, successivley decreasing charge, successively increasing and decreasing charge, or successively decreasing and increasing charge, and wherein the mode of each phase is configured as anodic or cathodic, and wherein a shape of the electrical pulses of the stimulation waveform for each phase is configured based on the pulse amplitude parameter, the phase mode, and the phase type.
Clause 5: The method of clause 4, wherein the stimulation waveform comprises a first phase and a second phase, the first phase and second phase having different phase types and different phase modes, wherein the number of phases, the phase types, and the phase modes for each phase are configured based on the phase parameters.
Clause 6: The method of clause 5, wherein the phase mode of the first phase is anodic and the phase mode of the second phase is cathodic, and wherein the first phase corresponds to a charge phase and the second phase is a discharge phase to discharge charge build up from the first phase.
Clause 7: The method of clause 6, wherien a phase type of the first phase is one of successively increasing charge, successivley decreasing charge, successively increasing and decreasing charge, or successively decreasing and increasing charge and a phase type of the second phase is different from the phase type of the first phase.
Clause 8: The method of clause 7, wherein successive time periods between electrical pulses of the stimulation waveform increase in time through the discharge phase to avoid action potential (AP) generation.
Clause 9: The method of any of clauses 1-8, further comprising: generating, by the IPG, a plurality of electrical pulses according to the programming data; and applying the generated electrical pulses to dorsal fibers of the spinal cord.
Clause 10: The method of clause 9, wherein the stimatulation waveform is configured to mitigate pain of the patient by suppressing nociceptive projection neurons in the superficial lamina of the spinal dorsal horn.
Clause 11: The method of any of clauses 1-10, wherein the electrical pulses of the stimulation wveform comprise plurality of electrical pulses, the plurality of electrical pulses comprising a first set of electrical pulses corresponding to a first phase of the stimulation waveform and a second set of electrical pulses corresponding to a second phase of the stimulation waveform, wherein the first set of electrical pulses comprise anodic pulses and the second set of electrical pulses comprise cathodic pulses.
Clause 12: The method of clause 11, wherein a pulse amplitude of the anodic pulses increases over time and a pulse amplitude of the cathodic pulses decreases over time.
Clause 13: The method of clause 11, wherein the pulse amplitude of the anodic pulses increases over time and the pulse amplitude of the cathodic pulses decreaes and then increases over time.
Clause 14: The method of clause 11, wherein the pulse amplitude of the anodic pulses increases over time and the pulse amplitude of the cathodic pulses increaes and then decreases over time.
Clause 15: The method of any of clauses 1-14, wherein a pulse amplitude of the stimulation waveform changes sinusoidally, linearly, or exponentially over time.
Clause 16: The method of any of clauses 1-15, wherein the electical pulses are configured to treat a medical condition of the patient.
Clause 17: The method of any of clauses 1-16, wherein the medical condition comprises pain of the patient.
Clause 18: The method of any of clauses 1-17, wherein the electrical pulses are configured to suppress projection neurons in a dorsal horn of a spine of the patient.
Clause 19: A clinician programmer for programming a stimulation waveform according to any preceding clause.
Clause 20: A non-transitory computer-readable storage medium storing instructions that, when executed by one or more processors, cause the one or more processors to perform operations according to any of clauses 1-18.
Clause 21: A clinician programmer for programming a stimulation waveform according to any of FIGS. 6-13.
Clause 22: A non-transitory computer-readable storage medium storing instructions that, when executed by one or more processors, cause the one or more processors to perform operations according to any of FIGS. 6-13.
Clause 23: a method comprising steps according to any of FIGS. 6-13.

Although the aspects of the present disclosure and their advantages have been described in detail, it should be understood that various changes, substitutions and alterations can be made herein without departing from the spirit of the disclosure as defined by the appended claims. Moreover, the scope of the present application is not intended to be limited to the particular implementations of the process, machine, manufacture, composition of matter, means, methods and processes described in the specification. As one of ordinary skill in the art will readily appreciate from the present disclosure, processes, machines, manufacture, compositions of matter, means, methods, or operations, presently existing or later to be developed that perform substantially the same function or achieve substantially the same result as the corresponding aspects described herein may be utilized according to the present disclosure. Accordingly, the appended claims are intended to include within their scope such processes, machines, manufacture, compositions of matter, means, methods, or operations.

What is claimed:

1. A method of providing a neurostimulation therapy to a patient, comprising:
receiving, by one or more processors of a clinician programmer device, inputs via a graphical user interface, the inputs corresponding to a set of parameters for configuring a stimulation waveform such that the waveform parameters and the phase parameters configure electrical properties of electrical pulses of the stimulation waveform and a shape of the stimulation waveform, wherein (a) the stimulation waveform comprises repetition of a burst of electrical pulses of one or two phases of selectable anodic or cathodic polarities based on respective ones of the inputs; (b) respective pulses of the one or two phases are separated by a time gap; (c) the amplitude of respective pulses within the one or two phases vary in manner that is increasing, decreasing, or combination thereof based on respective ones of the inputs; and (d) the electrical pulses of the one or two phases are charge limited such that the stimulation waveform is sub-threshold;
generating, by the one or more processors, programming data based on the one or more inputs, wherein the programming data is configured to control generation of a stimulation waveform based on the set of parameters; and
transmitting, by the one or more processors, the programming data to an implantable pulse generator (IPG).

2. The method of claim 1, wherien the waveform parameters comprise a pulse amplitude parameter defining maximum amplitude of the electrical pulses, a pulse width parameter defining a duration of each electrical pulse, an inter-pulse interval parameter defining a duration of time between each electrical pulse, a maximum number of pulses definition parameter defining a maximum number of pulses in the stimulation waveform, a waveform duration parameter defining a maximum duration of the stimulation waveform, or a combination thereof.

3. The method of claim 1, wherein the of electrical pulses are charge limited to be sub-threshold.

4. The method of claim 1, wherien the waveform parameters comprise a number of phases parameter defining a number of phases of the stimualtion waveform, a phase type parameter to configure a phase type for each phase of the stimulation waveform, a phase mode parameter to configure a mode for each phase of the stimulation waveform, wherein the phase type is one of successively increasing charge, successivley decreasing charge, successively increasing and decreasing charge, or successively decreasing and increasing charge, and wherein the mode of each phase is configured as anodic or cathodic, and wherein a shape of the electrical pulses of the stimulation waveform for each phase is configured based on the pulse amplitude parameter, the phase mode, and the phase type.

5. The method of claim 4, wherein the stimulation waveform comprises a first phase and a second phase, the first phase and second phase having different phase types and different phase modes, wherein the number of phases, the phase types, and the phase modes for each phase are configured based on the phase parameters.

6. The method of claim 5, wherein the phase mode of the first phase is anodic and the phase mode of the second phase is cathodic, and wherein the first phase corresponds to a charge phase and the second phase is a discharge phase to discharge charge build up from the first phase.

7. The method of claim 6, wherien a phase type of the first phase is one of successively increasing charge, successivley decreasing charge, successively increasing and decreasing charge, or successively decreasing and increasing charge and a phase type of the second phase is different from the phase type of the first phase.

8. The method of claim 7, wherein successive time periods between electrical pulses of the stimulation waveform increase in time through the discharge phase to avoid action potential (AP) generation.

9. The method of claim 1, further comprising:
generating, by the IPG, a plurality of electrical pulses according to the programming data; and
applying the generated electrical pulses to dorsal fibers of the spinal cord.

10. The method of claim 9, wherein the stimatulation waveform is configured to mitigate pain of the patient by suppressing nociceptive projection neurons in the superficial lamina of the spinal dorsal horn.

11. The method of claim 1, wherein the electrical pulses of the stimulation wveform comprise plurality of electrical pulses, the plurality of electrical pulses comprising a first set of electrical pulses corresponding to a first phase of the stimulation waveform and a second set of electrical pulses corresponding to a second phase of the stimulation waveform, wherein the first set of electrical pulses comprise anodic pulses and the second set of electrical pulses comprise cathodic pulses.

12. The method of claim 11, wherein a pulse amplitude of the anodic pulses increases over time and a pulse amplitude of the cathodic pulses decreases over time.

13. The method of claim 11, wherein the pulse amplitude of the anodic pulses increases over time and the pulse amplitude of the cathodic pulses decreaes and then increases over time.

14. The method of claim 11, wherein the pulse amplitude of the anodic pulses increases over time and the pulse amplitude of the cathodic pulses increaes and then decreases over time.

15. The method of claim 1, wherein a pulse amplitude of the stimulation waveform changes sinusoidally, linearly, or exponentially over time.

16. The method of claim 1, wherein the electical pulses are configured to treat a medical condition of the patient.

17. The method of claim 16, wherein the medical condition comprises pain of the patient.

18. The method of claim 1, wherein the electrical pulses are configured to suppress projection neurons in a dorsal horn of a spine of the patient.
